(19) United States
(12) Patent Application Publication
(10) Pub. No.: US 2023/0011344 A1
(43) Pub. Date: Jan. 12, 2023

(54) DEVICE FOR DIFFUSING VOLATILE SUBSTANCES

(71) Applicant: ZOBELE HOLDING SPA, TRENTO (IT)

(72) Inventors: Marina TRIAS LAFUENTE, BARCELONA (ES); Alba GRAUS FERRER, BARCELONA (ES); Fernando ALFONSO GALLEGO, BARCELONA (ES)

(73) Assignee: ZOBELE HOLDING SPA, TRENTO (IT)

(21) Appl. No.: 17/786,401

(22) PCT Filed: Dec. 18, 2020
(86) PCT No.: PCT/EP2020/086971

§ 371 Date: Jun. 16, 2022

(30) Foreign Application Priority Data
Dec. 18, 2019 (ES) P201931122

(51) Int. Cl. A61L 9/12

(52) U.S. Cl. CPC A61L 9/127

(57) ABSTRACT

The device for diffusing volatile substances, which having a container that includes a mouthpiece, the container containing a liquid with volatile substances; and a wick that is impregnated with said liquid contained in the container, which also includes a base located around the mouthpiece of the container, the wick being fixed in the interior of said base. It makes it possible to avoid leaks and to ensure the correct dosage and evaporation of the formulation that is inside the container.

The present invention relates to a device for diffusing volatile substances, in particular to an inverted positioning static air freshener.

### BACKGROUND OF THE INVENTION

There are inverted positioning static air freshener on the market. As they are products in an inverted position, the difficulty of the solutions consists in the correct dosage of the formulation, both at constant temperature and in cycles of temperature change. In existing products, this solution consists of an intermediate wick fixed to a container by means of a stop.

By placing the container on its base, this intermediate wick comes into contact with a secondary wick. The intermediate wick functions as a dispenser for the formulation, and the secondary wick as an evaporation surface.

This construction allows that when there is an overdose of the formulation due to an increase in temperature, it is returned to the container automatically when it cools.

A drawback of this type of inverted positioning static air fresheners is that the intermediate wick forms part of the fragrance container, so that good contact between the intermediate wick and the secondary wick cannot be ensured.

Furthermore, another drawback is that dripping may occur at the time of activation, and the product is for single use only.

### DESCRIPTION OF THE INVENTION

With the device for diffusing volatile substances of the invention, the aforementioned drawbacks are solved, presenting other advantages that will be described below.

The device for diffusing volatile substances according to the present invention comprises:

a container that includes a mouthpiece, the container containing a liquid with volatile substances and

a wick that is impregnated with said liquid contained in the container,

and it also comprises a base located around the mouthpiece of the container, the wick being fixed inside said base.

Furthermore, advantageously, the mouthpiece of the container comprises a cap provided with a hole, the cap being in contact with the wick.

According to a preferred embodiment, the base is a hollow body provided with at least one side hole.

Preferably, the base is removably fixed to said container, in any suitable way, for example, by means of a thread or by pressure.

Furthermore, the container may comprise a membrane that allows air to escape, but prevents the liquid from escaping.

If desired, the mouthpiece of the container may comprise a dosing pad.

Preferably, the cap is positioned around the outside of the container mouthpiece, for example screwed onto it.

According to a preferred embodiment, the membrane is located at an opposite end of the mouthpiece of the container.

Furthermore, in the position of use of the device for diffusing volatile substances according to the present invention, the mouthpiece of the container is located at the lower end of the container, and the membrane is located at the upper end of the container.

The device for diffusing volatile substances according to the present invention has, among others, the following advantages:
Avoids leaks;
Ensures the correct dosage and evaporation of the formulation that is inside the container;
The wick incorporates a system for fixing and easy releasing the subassembly without the need for the user to come into contact with the impregnated wick.
Allows the reuse of parts.

### BRIEF DESCRIPTION OF THE DRAWINGS

In order to better understanding what has been stated, some drawings are attached in which, schematically and only as a non-limiting example, a practical case of embodiment is represented.

FIG. 1 is an elevation section view of the device for diffusing volatile substances according to the present invention.

### DESCRIPTION OF A PREFERRED EMBODIMENT

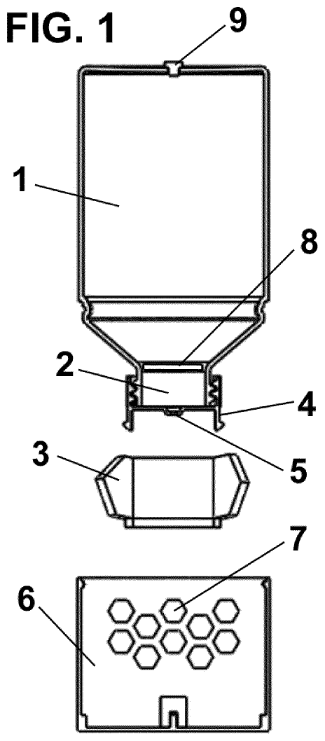

As shown in FIG. 1, the device for diffusing volatile substances according to the present invention comprises a container 1 inside which a liquid is housed containing the volatile substances that will be diffused into the environment.

Said volatile substances may be used for perfuming the environment, using the device as an air freshener, or against insects, using the device as an insecticide.

The container 1 comprises a mouthpiece 2 through which the liquid comes out, which is impregnated in a wick 3, from which it diffuses into the environment.

Around said mouthpiece 2 a cap 4 is placed that is in contact with the wick 3. If desired, the wick 3 can be joined to the cap 4. The function of the cap 4 is to close the mouthpiece 2, except for a hole 5 of the cap 4, where the liquid comes out to impregnate the wick 3.

Said cap 4 is placed in the mouthpiece 2 in any suitable way, for example, by means of a thread, as shown in FIG. 1.

The device for diffusing volatile substances according to the present invention also comprises a base 6 provided with a plurality of holes 7.

Said base 6 is preferably a hollow body that is removably connected to the container 1, for example, by means of a thread or pressure, and houses inside the wick 3 and the area of the mouthpiece 2 of the container 1, as can be deduced from FIG. 1.

Optionally, the container 1 can comprise a dosing pad 8 to control the dosage of the liquid to the wick 3. This dosing pad 8 can be placed close to the mouthpiece 2 of the container 1, although it is optional and not essential.

Furthermore, the container 1 of the diffusion device according to the present invention also comprises a membrane 9, which is permeable to air, but impermeable to liquids.

As can be seen in FIG. 1, the diffusion device according to the present invention is used in an inverted position, that is, with the mouthpiece 2 of the container 1 at the bottom, the device being supported on a surface by means of base 6.

In its position of use, the membrane 9 is located at the opposite end to the mouthpiece 2 of the container 1, that is to say, in the upper part of the container 1.

The operation of the device for diffusing volatile substances according to the present invention is very simple. Simply the container 1 is placed in an inverted position on the base 6 and, in this position, the liquid comes out through the hole 5 of the cap 4 and impregnates the wick 3.

The wick 3 impregnated with the liquid, which is fixed inside the base 6, shall diffuse the volatile substances into the environment, leaving the base 6 through the holes 7 that are located on the side wall of the base 6.

The membrane 9 located in the upper part of the container 1, in the position of use represented in FIG. 1, allows the air inside the container 1 to escape from it, but prevents the leakage of the liquid, which will only come out of the container 1 through the hole 5 of the cap 4.

In this way, as the device for diffusing volatile substances is subject to changes in temperature, the membrane 9 makes it possible to release pressure and thus avoid overdosing the liquid.

Despite the fact that reference has been made to a specific embodiment of the invention, it is obvious to a person skilled in the art that the device for diffusing volatile substances described is susceptible to numerous variations and modifications, and that all the mentioned details can be substituted by others technically equivalent, without departing from the scope of protection defined by the appended claims.

1. A device for diffusing volatile substances, comprising:
a container including a mouthpiece, the container containing a liquid with volatile substances; and
a wick that is impregnated with said liquid contained in the container,
characterized in that it also comprises a base located around the mouthpiece of the container, the wick being fixed inside said base.

2. The device for diffusing volatile substances according to claim 1, wherein the mouthpiece of the container comprises a cap provided with a hole, the cap being in contact with the wick.

3. The device for diffusing volatile substances according to claim 1, wherein the base is a hollow body provided with at least one side hole.

4. The device for diffusing volatile substances according to claim 1, wherein the base is removably fixed to said container.

5. The device for diffusing volatile substances according to claim 1, wherein the container comprises a membrane that allows air to escape, but prevents the liquid from escaping.

6. The device for diffusing volatile substances according to claim 1, wherein the mouthpiece of the container comprises a dosing pad.

7. The device for diffusing volatile substances according to claim 2, wherein the cap is placed around the outside of the mouthpiece of the container.

8. The device for diffusing volatile substances according to claim 5, wherein the membrane is located at an opposite end of the mouthpiece of the container.

9. The device for diffusing volatile substances according to claim 1, wherein, in its position of use, the mouthpiece of the container is positioned at the lower end of the container.

10. The device for diffusing volatile substances according to claim 5, wherein, in its position of use, the membrane is placed at the upper end of the container.

11. The device for diffusing volatile substances according to claim 6, wherein, in its position of use, the membrane is placed at the upper end of the container.

12. The device for diffusing volatile substances according to claim 7, wherein, in its position of use, the membrane is placed at the upper end of the container.

13. The device for diffusing volatile substances according to claim 8, wherein, in its position of use, the membrane is placed at the upper end of the container.
